(19) United States
(12) Patent Application Publication
(10) Pub. No.: US 2025/0281252 A1
(43) Pub. Date: Sep. 11, 2025

(54) METHOD FOR CONTROLLING, BY DECREASING THE IMPARTED SPEED OR POWER, A SLAVE DEVICE CONTROLLED BY A MASTER DEVICE IN A ROBOTIC SYSTEM FOR MEDICAL OR SURGICAL TELEOPERATION, AND RELATED ROBOTIC SYSTEM

(71) Applicant: MEDICAL MICROINSTRUMENTS, INC., Wilmington, DE (US)

(72) Inventors: Antonio DI GUARDO, Pisa (IT); Emanuele RUFFALDI, Pisa (IT); Matteo TANZINI, Pisa (IT); Michael John PROCTOR, Pisa (IT); Massimiliano SIMI, Pisa (IT)

(21) Appl. No.: 18/851,674

(22) PCT Filed: Mar. 28, 2023
(86) PCT No.: PCT/IB2023/053073

§ 371 Date: Sep. 26, 2024

(30) Foreign Application Priority Data
Mar. 31, 2022 (IT) 102022000006332

(51) Int. Cl. A61B 34/37; B25J 9/16; G16H 40/63

(52) U.S. Cl. CPC A61B 34/37; B25J 9/1648; B25J 9/1651; G16H 40/63

(57) ABSTRACT

A method for controlling a slave device during teleoperation is performed by a robotic system. The robotic system includes a master device movable by an operator, and a slave device having a surgical instrument controllable by the master device. The method defines a nominal target pose in a workspace of the slave device, modifies the nominal target pose to obtain a modified target pose, and controls motion of the slave device in the workspace so that the slave follows the modified target pose. Modifying the nominal target pose includes decreasing a translational speed module of the modified target pose relative to speed of the nominal target pose, and/or decreasing instantaneous power or energy imparted to the slave device, according to a transfer function dependent on the instantaneous speed/instantaneous power of the master device and/or the distance between a current position and the nominal target pose of the slave device.

### TECHNOLOGICAL BACKGROUND OF THE INVENTION

### Field of Application

The present invention relates to a method and system for controlling a teleoperation carried out by means of a robotic system for medical or surgical teleoperation.

In particular, the invention relates to a method for controlling, by means of passivation techniques (i.e., controlled reduction of speed or power imparted), a slave device controlled by a master device movable by an operator, in a robotic system for medical or surgical teleoperation.

### Description of the Prior Art

In the context of master-slave robotic systems for medical or surgical teleoperation, systems are known with master devices which are not mechanically constrained to a “master controller” station of the robotic system, i.e., “wheel” master (or “mechanically ungrounded”, “mechanically unconstrained”), or of the type as shown for example in WO-2019-020407, WO-2019-020408. WO-2019-020409 in the name of the same Applicant.

In all master-slave robotic systems for medical or surgical teleoperation, and, in particular, in those with unconstrained master devices, mentioned above, the operator moves the master device, during the teleoperation, and the control system allows the slave device to move for following/tracking the position and orientation of the master device.

Actually, during the aforesaid following/tracking, the drawback arises that the position and instantaneous orientation of the master device are not equal to those of the slave device. This problem stems from several causes, including:
1) presence of delays within the system, i.e., the time necessary for the propagation of the operator's signal from the tracking device to the control system, and then to the motors which actuate the movements of the slave device;
2) intrinsic limits of the actuation system, i.e., the fact that the motors actuating the movements of the slave device have a maximum working speed and acceleration;
3) mechanical resonance of the mechanical components of the robot; in this case the dynamics are “deliberately” slowed down to avoid hitting the robot, inducing any undesired vibrations.

If the instantaneous error (distance) between the actual pose of the slave device and the correct pose commanded by the master device (i.e., the pose which would be obtained in the absence of the aforesaid phenomena/imperfections) becomes too large, several problems occur including the following.
PROBLEM 1) When the operator stops, the robot is still moving. In this context:
   the operator perceives a delay between the action he/she has performed and the action performed by the robot;
   the operator tends to close the control cycle with his/her eye, and thus on his/her perception, affected by the aforesaid delay; in other words, the presence of a delay, does not allow the operator to position the slave device finely and precisely after a fast motion.
PROBLEM 2) When the operator changes direction quickly (for example, makes a circumference in space with the master device) the following additional drawbacks are determined:
   in changes of direction, due to the accumulated “movement delay”, the operator perceives that the slave device is still going “forward” while the master device is already going “backward”;
   at high instantaneous speeds (close to or higher than those of the actuation system) and curvilinear trajectories, distortions of the movement of the slave device are generated, since the slave device tends to converge to the master device along the shortest possible (instantaneous) trajectory, without taking into account the instantaneous trajectory made by the operator. In an extreme situation, a circular trajectory of the master device transforms into a square trajectory of the slave device.

The known solutions, in the technical field considered, do not allow satisfactorily solving the aforesaid problems and drawbacks.

Therefore, in the considered technical field of robotic systems for master-slave teleoperation (whether they have a constrained master or not) and related control methods, there is a strong need to control the enslaved motion of the slave device, depending on the master device, with expedients and based on control algorithms such as to solve or at least mitigate the aforesaid problems and drawbacks.

### SUMMARY OF THE INVENTION

It is an object of the present invention to provide a method for controlling, by means of passivation techniques, a slave device controlled by a master device and movable by an operator, which allows at least partially overcoming the drawbacks complained above with reference to the prior art, and responding to the aforementioned needs particularly felt in the technical field considered.

Such an object is achieved by a method according to claim 1.

Further embodiments of such a method are defined in claims 2-17.

It is also an object of the present invention to provide a robotic system for medical or surgical teleoperation, configured to be controlled by means of the aforesaid method. Such an object is achieved by a system according to claim 18.

Further embodiments of such a system are defined by claim 19.

### BRIEF DESCRIPTION OF THE DRAWINGS

Further features and advantages of the method according to the invention will become apparent from the following description of preferred embodiments, given by way of non-limiting indication, with reference to the accompanying drawings, in which:

FIG. 1A shows a master-slave robotic system for medical or surgical teleoperation, according to an embodiment of the present invention;

FIG. 1B shows in more detail a master device and a slave device, included in the robotic system in FIG. 1, according to an embodiment of the present invention;

FIG. 2 shows, in a diagrammatic and simplified manner, an embodiment of a control method included in the present invention;

FIG. 3 shows a transfer function between a nominal slave device speed module (or input speed/velocity Vin) and a modified device speed module (or output speed/velocity Vout) according to an embodiment of the control method of the present invention;

FIG. 4 shows a relationship between a nominal slave device speed module (or input speed/velocity Vin) and scale multiplication factor according to an embodiment of the control method of the present invention;

FIG. 5 shows a modified target pose trajectory of the slave device with respect to a respective nominal trajectory (or “virtual target”), determined by a control which does not include a passivation action as provided by the present method;

FIG. 6 shows a modified target pose trajectory of the slave device with respect to a respective nominal trajectory (or “virtual target”), determined by a control according to the method of the present invention which includes a passivation action;

FIG. 7 shows an example of time evolution of the controlled speed module of the slave device with respect to a nominal speed module in a situation corresponding to that in FIG. 5;

FIG. 8 shows an example of time evolution of the controlled speed module of the slave device with respect to a nominal speed module in a situation corresponding to that in FIG. 6;

FIGS. 9A and 9B respectively illustrate the actions carried out by a “passivator” block and a control flow diagram, according to an implementation option of the method of the present invention;

FIGS. 10A and 10B respectively illustrate the actions carried out by a “passivator” block and a control flow diagram, according to another implementation option of the method of the present invention.

### DETAILED DESCRIPTION

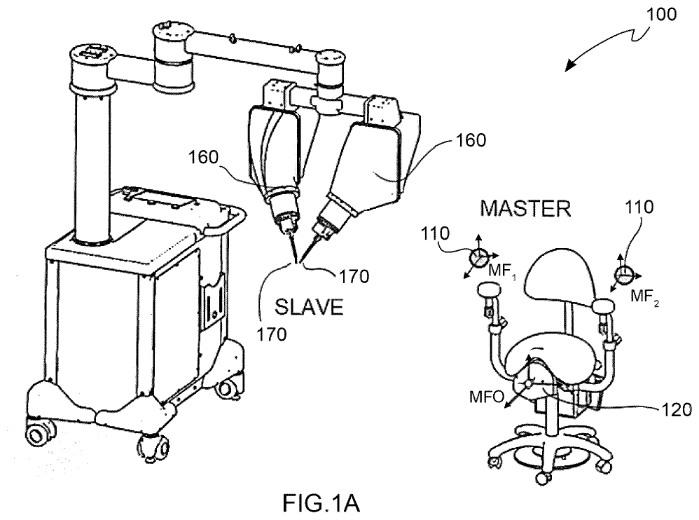

With reference to FIGS. 1-10, a method for controlling a slave device during a teleoperation performed by means of a robotic system 100 for medical or surgical teleoperation is described.

The aforesaid robotic system comprises at least one master device 110 adapted to be moved by an operator 150, and at least one slave device comprising a surgical instrument 170 adapted to be controlled by the master device.

The master device 110 is preferably a “wheel” type master device, without force feedback, for mono-lateral teleoperation. For example, therefore, the master device can be a master mechanically constrained to an operating console and at the same time be of the “wheel” type without force feedback, for single-sided teleoperation.

The master device 110 is preferably a master device of a type which is mechanically unconstrained to the operating console.

The method comprises the steps of defining a nominal target pose in a workspace of the slave device (corresponding to a respective pose of the master device in a workspace of the master device), modifying the nominal target pose to obtain a modified target pose of the slave device, and controlling the motion of the slave device in the slave device workspace so that the slave device is configured to follow the aforesaid modified target pose during the teleoperation.

The aforesaid step of modifying the nominal target pose to obtain the modified target pose comprises decreasing the translational speed module of the modified target pose, with respect to the speed of the nominal target pose, and/or decreasing the instantaneous power or energy imparted by the master device to the slave device, according to a respective transfer function.

Such a transfer function depends on the instantaneous speed of the master device and/or the instantaneous power or energy of the master device and/or the distance between a current position of the slave device and the nominal target pose of the slave device.

In accordance with an embodiment of the method, the aforesaid step of modifying the nominal target pose to obtain a modified target pose of the slave device causes a controlled loss of positional coherence between the master device and the slave device and reduces the delay of the slave device motion, perceived by the operator during the teleoperation, with respect to the master device motion.

It should be noted that, although such a loss of coherence may appear disadvantageous at first sight, it provides a surprisingly useful technical effect, especially in terms of reducing the delay of the slave device motion, with respect to the motion imparted to the master device, perceived by the operator during the teleoperation, for example in the event of changes in direction of such a motion.

According to an embodiment of the method, the aforesaid step of modifying the nominal target pose comprises decreasing the translational speed module of the modified target pose.

According to an implementation option, the translational speed of the modified target pose is expressed with reference to a system of orthogonal Cartesian coordinates in the slave device workspace.

According to another implementation option, the translational speed of the modified target pose is expressed with reference to coordinates of a space of the joints of the slave device.

Therefore, in accordance with the aforesaid implementation options, the method provides the effect of relating the speeds of the control points or relating the speeds to the joints.

In this second case, the joints referred to are joints which allow controlling the degrees of freedom of the slave surgical instrument 170, for example:
joints of the slave articulated surgical instrument which control respectively degrees of freedom of yaw rotation and pitch rotation;
joint of the slave articulated surgical instrument which controls a degree of freedom of roll rotation around a shaft of the surgical instrument;
joints which control three translational degrees of freedom X, Y, Z, and which are typically arranged at a robotic manipulator 160 upstream of the slave surgical instrument 170 (where the master device controls the robotic manipulator 160 associated with each slave surgical instrument 170).

According to an implementation option, the transfer function which modifies the translational speed of the modified target pose manages each of the speed components into which the speed is decomposed (e.g., components associated with a decomposition in accordance with the coordinates of the chosen reference coordinate system) in a mutually independent manner.

According to possible embodiments, the method is applied to a mono-lateral or bilateral teleoperation from the master device to the slave surgical instrument.

The embodiment which applies to a mono-lateral teleoperation includes applying the method to a situation in which there is no feedback on the master device (as opposed to what is included in a bilateral teleoperation).

In accordance with an embodiment of the method, the transfer function which modifies the translational speed of the modified target pose depends exclusively on the speed of the master device.

In this case, a continuous and monotonous non-decreasing function is used, defined as a linear function, for speed values below a predefined threshold speed value. and a non-linear function for speed values above said threshold speed value.

In the linear section of the function, the modified target pose speed module of the slave device remains unchanged with respect to the nominal target pose speed module.

In the non-linear section of the function, the modified target pose speed module of the slave device is reduced with respect to the nominal target pose speed module.

According to a particular implementation option, the aforesaid threshold speed value is between 0.015 m/s and 0.025 m/s.

In accordance with an implementation option, the aforesaid non-linear section of the speed transfer function has a trend tending to a horizontal asymptote defining the maximum speed of the slave device target.

According to an implementation option, the aforesaid maximum speed value of the slave device target corresponds to the maximum speed module being reachable by the slave device itself.

According to an implementation option, the maximum speed of the slave device target is tunable.

In accordance with an embodiment of the method, the transfer function which modifies the translational speed of the modified target pose is dependent on the nominal target pose speed and on the virtual distance between the position of the nominal target pose and the current position of the slave device.

According to an implementation option, the aforesaid transfer function is a virtual distance transfer function, and is a continuous and monotonous non-decreasing function. Such a function is defined as a linear function, for virtual distance values below a predefined threshold distance value, and a non-linear function for virtual distance values above said threshold distance value.

In the linear section of the function, the modified target pose speed module of the slave device remains unchanged with respect to the nominal target pose speed module.

In the non-linear section of the function, the modified target pose speed module of the slave device is reduced, with respect to the nominal target pose speed module, by an amount given by a transfer function of said virtual distance.

According to a particular implementation option, the aforesaid threshold distance value is between 0.5 mm and 5 mm.

In accordance with an implementation option, the virtual distance transfer function (non-passivated virtual distance) is a continuous, monotonous non-decreasing function, having the value “virtual distance+maximum distance” as an asymptote, where the parameter “maximum distance” is a tunable parameter defining a maximum allowed virtual distance between the modified target pose and the slave device position.

According to a particular implementation option, the aforesaid maximum virtual distance value is between 0.5 mm and 5 mm.

With reference to the aforementioned “poses” of the master and slave devices, it should be noted that, for the purposes of the present explanation, each “pose” is to be understood as characterized by respective values of the degrees of freedom of the slave device.

Typically, such degrees of freedom comprise 7 degrees of freedom, of which three degrees of freedom of translation (X, Y, Z), three degrees of freedom of rotation (for example, the aforementioned “roll”, “pitch”, “yaw”) and one degree of freedom of opening/closing (“grip”).

Thus, a “pose” is defined by respective values of the aforesaid degrees of freedom, and a velocity (i.e., speed) associated with a pose refers to a velocity (i.e., speed) of the temporal evolution of a respective degree of freedom; a translational speed refers to a translational speed in the coordinate system of the translational degrees of freedom X, Y, Z.

Still with reference to poses, the following definitions of “master pose”, “slave pose”, “nominal target pose”, “modified target pose” are used in the present description.

The “master pose” is the current pose of the master device in the reference coordinate system of a master device workspace (also defined in this description and in the figures as “master space”, comprising, for example, a space defined by a tracking mechanism included in the robotic system).

The “slave pose”: is the current pose of the slave device in the reference coordinate system of a slave device workspace (also defined in this description and in the figures as “slave space”).

The “nominal target pose” (also sometimes defined in the following as “proxy pose”) is the master device pose mapped in the slave device workspace; it is so defined because it is the pose that should be followed by the slave device under “nominal” conditions, i.e., in the absence of further control mechanisms or processing.

It should be noted that the determination of the “nominal target pose” depends solely on translation offsets between the centers of the master and slave reference coordinate systems and the application of the scale factor on the translations. Translation offsets can be defined for example in alignment steps, or by a direct intervention of the operator, or deriving from the action of usability algorithms.

The “modified target pose” (also sometimes defined in the following as the “target pose”) is the reference pose of the slave device, i.e., the pose to which the slave device must converge following the actuation governed by the control system. This pose can in principle coincide with the nominal target pose, but can also differ therefrom if there are reasons to modify it, by means of specific additional control actions and related algorithms.

In the present description, the modification of the nominal target pose (proxy pose), to obtain the modified target pose (target pose) is performed for example based on information on the current position of the slave device, so as to reduce the delays perceived by the operator between the motion of the slave device and the motion imparted to the master device.

Such a modification can be obtained, for example (as will be further illustrated below), by inserting an additional translation offset between proxy pose and target pose.

A robotic system 100 for medical or surgical teleoperation, according to the present invention is described below.

The robotic system comprises at least one master device 110 adapted to be moved by an operator 150 and at least one slave device comprising a surgical instrument 170 adapted to be controlled by the master device.

The robotic system further comprises a control unit configured to control the slave device, during a teleoperation, based on movements of the master device.

The control unit is further configured to carry out the following actions: defining a nominal target pose in a workspace of the slave device, corresponding to a respective pose of the master device in a workspace of the master device: modifying the aforesaid nominal target pose to obtain a modified target pose of the slave device; controlling the motion of the slave device in the slave device workspace so that the slave device is configured to follow the aforesaid modified target pose during a teleoperation.

In the aforesaid step of modifying the nominal target pose to obtain the modified target pose, the control unit is configured to decrease the translational speed module of the modified target pose (with respect to the speed of the nominal target pose), or to decrease the instantaneous power or energy imparted by the master device to the slave device, according to a respective transfer function dependent on the instantaneous speed of the master device and/or the instantaneous power or energy of the master device and/or the distance between a current position of the slave device and the nominal target pose of the slave device.

According to several possible embodiments of the robotic system, the control unit is configured to carry out a method for controlling a slave device according to any one of the embodiments previously claimed.

Still with reference to FIGS. 1-10, further details will be provided below, by way of non-limiting example, with reference to some particular embodiments of the method and of the robotic system according to the present invention.

As previously noted, the “master pose” of the master device, mapped in the slave device space and appropriately scaled by a possible scale factor, uniquely defines a “nominal target pose” (“proxy pose”) of the slave device which is used as a reference by the control unit for controlling the slave device.

As already illustrated, the present method includes modifying the aforesaid nominal target pose of the slave device in such a way to reduce the delay perceived by the operator when executing his/her commands during the teleoperation itself, at the cost of losing the positional coherence between the master device and the slave device. Such a reduction is obtained by reducing the energy introduced by the operator in the slave system, i.e., by a technique which is defined here as “passivation”.

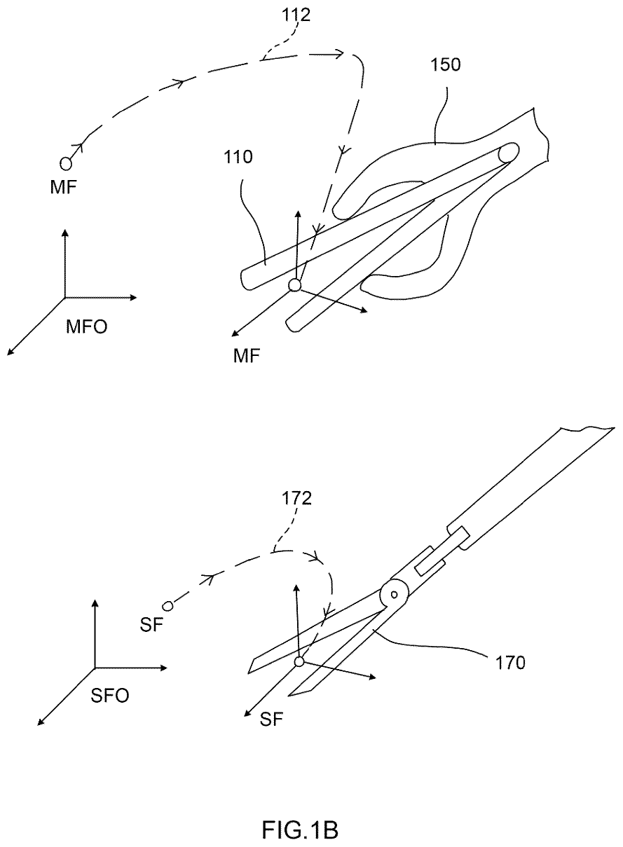

This is illustrated for example in FIG. 2, where the action of modifying the nominal target pose (indicated in FIG. 2 as “target slave−pre-passiv”) to reach the modified target pose (indicated in FIG. 2 as “target slave−post-passiv”) is carried out by the block indicated as “passivator”.

In an implementation option, the target pose modification occurs in such a way to reduce the power (or, equivalently, the energy) introduced in the system by the master device.

In an implementation option, the modification of the target pose of the slave device occurs by reducing the speed module of such a target pose of the slave device.

The speed of the slave device target pose can be expressed according to a system of Cartesian axes, or in the space of the joints of the robotic system.

In an implementation option, the passivation process occurs independently for each of the components in which the target pose speed vector of the slave device is decomposed.

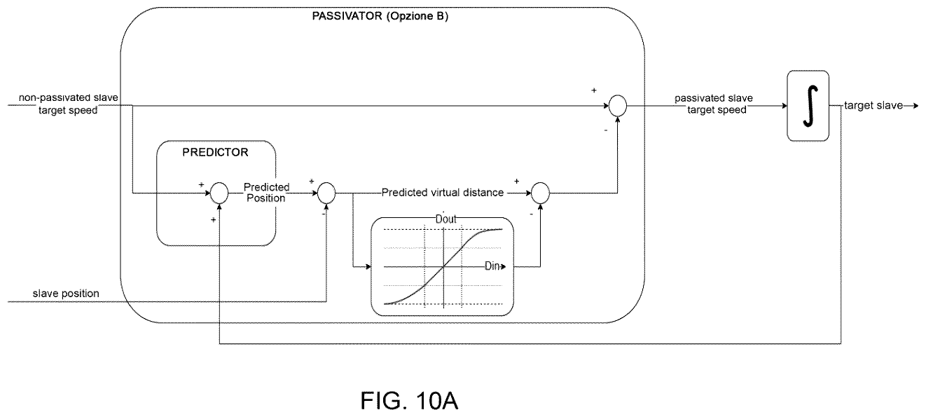

According to an implementation option (shown in FIGS. 9A and 9B as “option A”), the speed module after the passivation process depends on the speed module of such a target pose prior to the passivation process, through a speed transfer function.

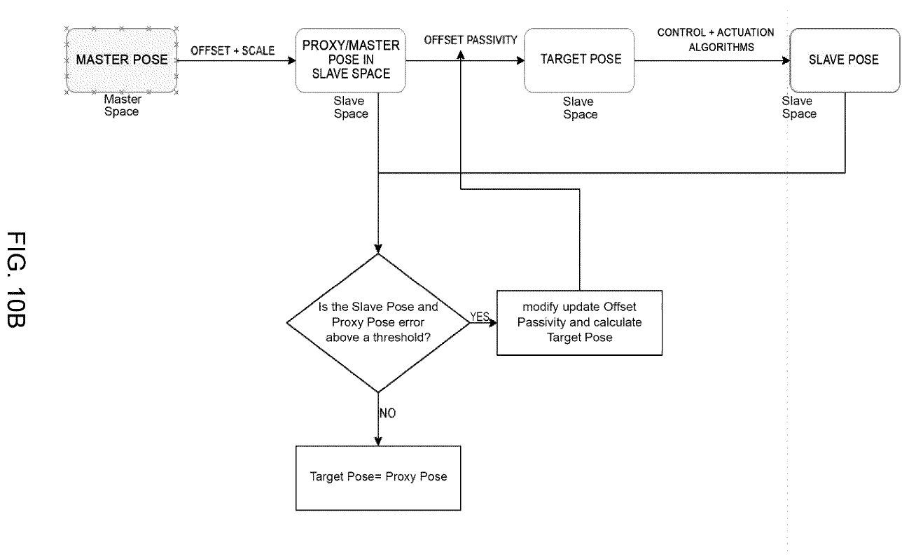

According to another implementation option (shown in FIGS. 10a and 10B as “option B”), the speed module after the passivation process depends on the speed module of such a target pose prior to the passivation process, and further on the position of the slave device.

In FIGS. 9 and 10, the nominal target pose speed (before passivation) is referred to as “non-passivated slave target speed”, while the modified target pose speed (after passivation) is referred to as “passivated slave target speed”.

In option A, the aforesaid speed transfer function is a linear function, for speed values below a predefined threshold speed value, in a linear section of the transfer function in which the target speed module of the slave device remains unchanged.

The aforesaid speed transfer function is instead a non-linear function for speed values above said threshold speed value, in a non-linear section of the transfer function in which the speed module of the target of the slave device is reduced based on the speed of the target of the non-passivated slave device itself.

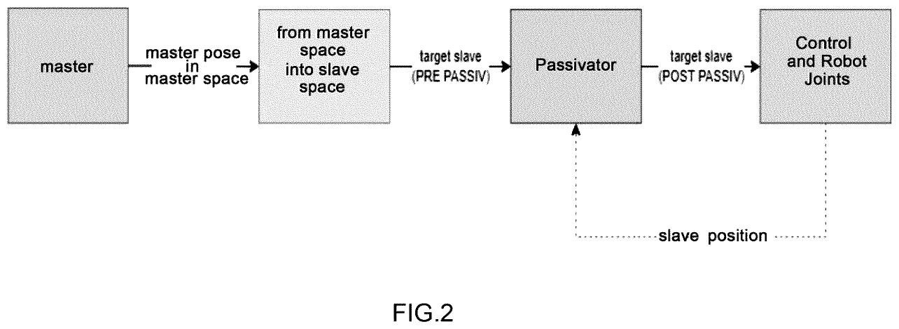

In accordance with an embodiment of the method (shown for example in FIG. 3), the aforesaid speed transfer function is a continuous, monotonous non-decreasing function.

According to an embodiment, the aforesaid non-linear section of the speed transfer function has a trend tending to an asymptote dependent on a maximum speed value of the target of the slave device.

According to an implementation option of the method, the aforesaid maximum speed value of the slave device corresponds to a maximum speed module value achievable by the slave device.

According to several possible implementation options, the aforesaid asymptote can be a horizontal asymptote or an oblique asymptote.

In particular, according to an implementation option, the aforesaid asymptote is a horizontal asymptote, placed at a speed value equal to the sum of the aforesaid maximum speed value of the slave device and an offset value (DELTA).

In accordance with an implementation option, the aforesaid offset value (DELTA) is null.

Therefore, in this case, the speed transfer function is such that the speed module of the slave device, when the speed of the master device increases beyond the threshold speed value, increases continuously but less than proportionally, so as to gradually reach the maximum speed of the slave device, which is never exceeded.

According to an implementation option, the aforesaid offset value (DELTA) is tunable.

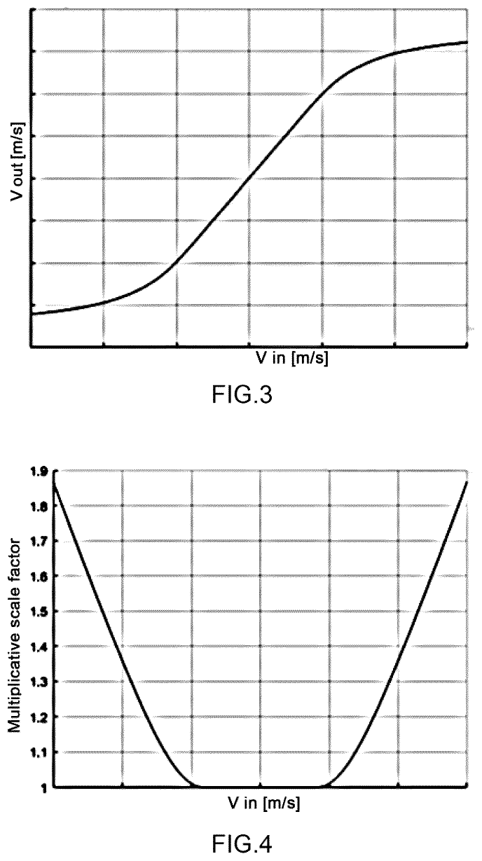

Option A can be interpreted geometrically as follows. Let an N scale factor be given, which determines the ratio between the magnitude of the movements of the master device and the slave device. The target pose speed of the slave device is thus obtained by multiplying the scale factor N by the instantaneous speed of the master device. The passivation operation described by an implementation option of option A is therefore equivalent to instantaneously multiplying the scale factor N by a multiplicative parameter of variable control and dependent on the speed of the master device itself. Such a control multiplication parameter (or “multiplicative scale factor”, as exemplarily shown in FIG. 4) has a value 1, when the master device speed module is less than the aforesaid threshold speed value, and grows substantially linearly monotonously non-decreasing as the slave device speed module changes.

According to an embodiment of the method, the aforesaid threshold speed value is between 0.015 m/s and 0.025 m/s, as a function of the speed limits of the joints of the slave system.

In particular, according to an implementation option, the aforesaid threshold speed value is 0.02 m/s.

According to the embodiment of the method based on the aforesaid option B (FIG. 10), the speed of the target of the slave device is a function of both the speed of the target of the slave device before the passivation itself, and of the virtual distance between the target of the non-passivated slave device and the slave device itself.

According to such an embodiment, the target speed of the passivated slave device (i.e., modified target pose speed) is equal to the nominal target pose speed (i.e., that of the non-passivated slave target) decreased by a transfer function dependent on the virtual distance between the slave device and the predicted slave target position assuming non-passivation (i.e., associated with the nominal target pose).

According to such an embodiment, such a virtual distance transfer function has the following features:
it is equal to zero if such a virtual distance is less than a limit virtual distance which, in an implementation option, is 2.5 mm;
for error values greater than such a limit virtual distance, the function behaves as a continuous function having asymptote equal to y=din−dmax, where din is the predicted virtual distance and dmax is a configurable parameter; in an implementation option, dmax is equal to 5 mm.

According to an embodiment, the method provides that, by virtue of the control determined by the aforesaid speed transfer function, the delay with which the slave device stops is reduced, following the stopping of the motion imparted by the surgeon to the master device.

According to an embodiment the method provides that, by virtue of the control determined by the aforesaid speed transfer function, the delay perceived by the user on the change of direction of the slave device is reduced with respect to the motion imparted by the surgeon to the master device.

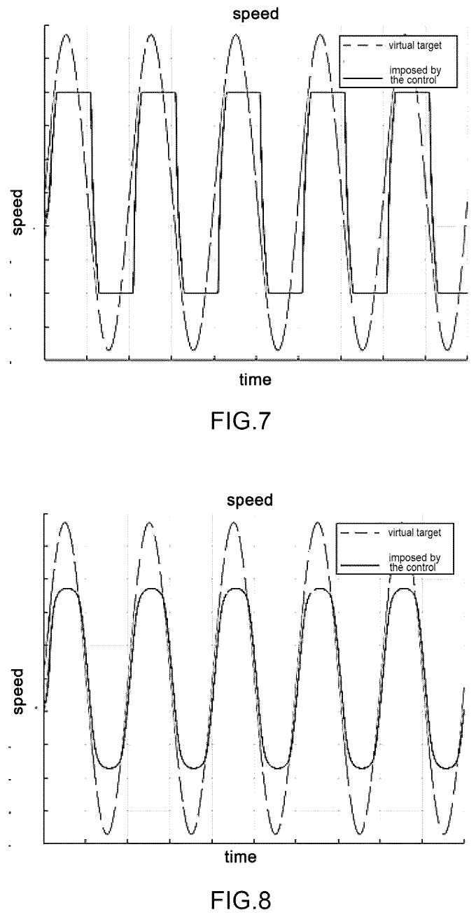

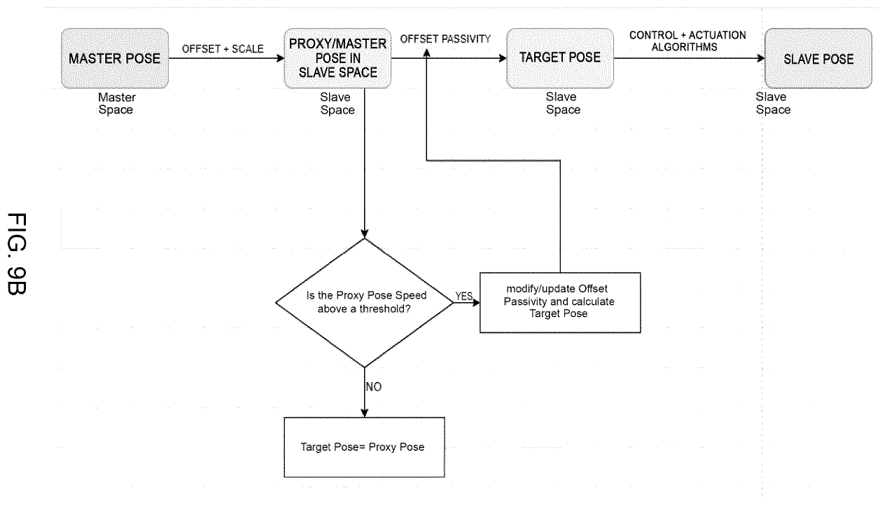

This is obtained by modifying the target pose with respect to the nominal target pose, as shown for example in FIGS. 6 and 8.

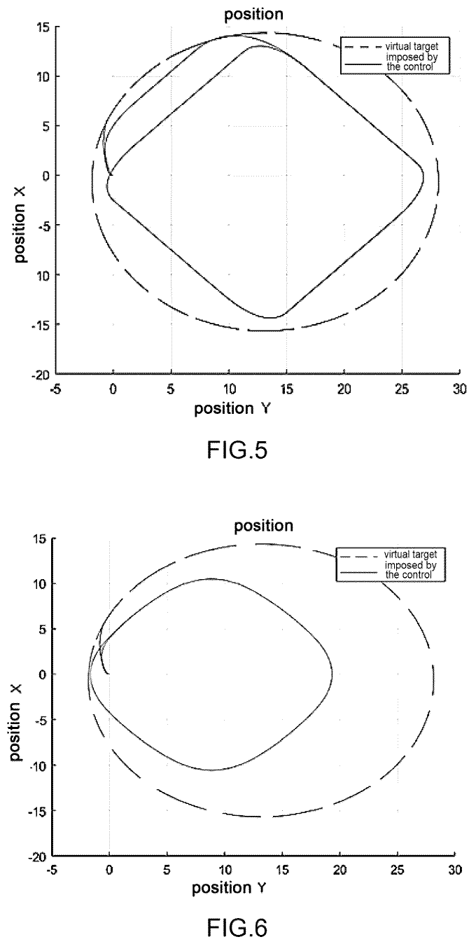

It should be noted that, in FIG. 5, a modified target pose trajectory of the slave device (indicated by a solid line referred to as “imposed by the control”) is illustrated with respect to a respective nominal trajectory (indicated by a dashed line referred to as “virtual target”), determined by a control which does not include a passivation action such as that included by the present method.

FIG. 6 shows a modified target pose trajectory of the slave device (indicated by a solid line referred to as “imposed by the control”) with respect to a respective nominal trajectory (indicated by a dashed line referred to as “virtual target”), determined by a control in accordance with an embodiment of the method of the present invention which includes a passivation action.

It should be noted that the examples in FIGS. 5 and 6 show by way of example two-dimensional trajectories, in the plane of the two coordinates X and Y, but the concept shown is easily and obviously extensible to the case of three-dimensional trajectories, in the space of the three coordinates X, Y, Z.

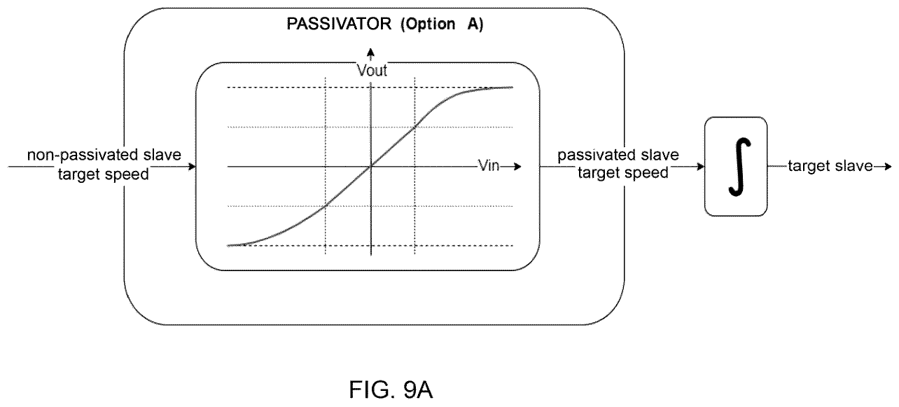

In FIG. 7, a time evolution example of the module of the controlled speed of the slave device (continuous line “imposed by the control”) with respect to the module of a nominal speed (dashed line “virtual target”) is shown in a situation corresponding to that in FIG. 5, in which a passivation action is not included.

In FIG. 8, a time evolution example of the module of the controlled speed of the slave device (continuous line “imposed by the control”) with respect to the module of a nominal speed (dashed line “virtual target”) is shown in a situation corresponding to that in FIG. 6, in which a passivation action is carried out according to an embodiment of the present method.

As already noted, according to an embodiment of the method, the action of the speed transfer function corresponds to a control with passivation, in which the amount of power and/or energy which is transferred to the control system of the slave device is limited in a controlled manner, with respect to a power and/or energy introduced by the operator, by means of motion of the master device, exceeding a certain power and/or energy threshold level.

As already noted, by virtue of the provision of three orthogonal translation slave joints X, Y, Z (e.g., motorized slides), it is possible to reduce, i.e., passivate, the module of the slave speed of said three orthogonal slave joints, to slow down the translation of the control point of the slave surgical instrument, avoiding, for example, forcing the joint(s) (e.g., slide(s)) responsible for the end-of-stroke translation. It is thus possible to intervene on the trajectory of the control point, as an evolution over time of the position of the poses of the control point. For example, if the system recognizes that the target pose would force the slave joint to reach the end-of-stroke, then the system determines the passivation of the slave trajectory, as previously described.

As already noted, in order to intervene on the dynamics of the slave device, the control parameters are intervened, whereby the “passivation” is understood here as a reduction in virtual power.

As can be seen, the objects of the present invention as previously indicated are fully achieved by the method disclosed above by virtue of the features described above in detail.

In order to meet contingent needs, those skilled in the art may make changes and adaptations to the embodiments of the method described above or can replace elements with others which are functionally equivalent, without departing from the scope of the following claims. Each of the features described above as belonging to a possible embodiment can be implemented irrespective of the other embodiments described.

### LIST OF REFERENCE SIGNS

100. Robotic system
110. Master device
112. Master device trajectory
120. Master device tracking field generator
150. Operator
160. Slave robotic manipulator
170. Slave surgical instrument
172. Slave surgical instrument trajectory
MFO. “Master frame origin” or origin of the master reference system
MF. “Master frame” or local master reference system
SFO. “Slave frame origin” or origin of the slave reference system
SF. “Slave frame” or local slave reference system

1. A method for controlling a slave device during a teleoperation performed by a robotic system for medical or surgical teleoperation, wherein said robotic system comprises at least one master device adapted to be moved by an operator, and at least one slave device comprising a surgical instrument adapted to be controlled by the master device, wherein the method comprises:
defining a nominal target pose in a workspace of the slave device, corresponding to a respective pose of the master device in a workspace of the master device;
modifying said nominal target pose to obtain a modified target pose of the slave device;
controlling motion of the slave device in the slave device workspace so that the slave device is configured to follow said modified target pose during the teleoperation;
wherein said step of modifying the nominal target pose to obtain the modified target pose comprises:
decreasing a translational speed module of the modified target pose, with respect to a speed of the nominal target pose, according to a transfer function dependent on an instantaneous speed of the master device and/or instantaneous power or energy of the master device and/or a distance between a current position of the slave device and the nominal target pose of the slave device,
and/or decreasing the instantaneous power or energy imparted by the master device to the slave device according to a transfer function dependent on the instantaneous speed of the master device and/or the instantaneous power or energy of master device and/or the distance between a current position of the slave device and the nominal target pose of the slave device.

2. A method according to claim 1, wherein said step of modifying the nominal target pose to obtain a modified target pose of the slave device causes a controlled loss of positional coherence between the master device and the slave device and reduces delay of the slave device motion, perceived by the operator during the teleoperation, with respect to the master device motion.

3. A method according to claim 1, wherein said step of modifying the nominal target pose comprises decreasing the translational speed module of the modified target pose, wherein the translational speed of the modified target pose is expressed with reference to an orthogonal Cartesian coordinate system in the slave workspace.

4. A method according to claim 1, wherein said step of modifying the nominal target pose comprises decreasing the translational speed module of the modified target pose, wherein the translational speed of the modified target pose is expressed with reference to coordinates of a space of joints of the slave device.

5. A method according to claim 3, wherein the transfer function that modifies the translational speed of the modified target pose manages each of speed components, into which the speed is decomposed, in a mutually independent manner.

6. A method according to claim 1, wherein the teleoperation is a single-sided or mono-lateral teleoperation from the master device to the slave surgical instrument.

7. A method according to claim 3, wherein the transfer function that modifies the translational speed of the modified target pose is solely dependent on the master device speed and is a continuous and monotonous non-decreasing function, defined as:
a linear function, for speed values below a predetermined threshold speed value, in which the modified target pose speed module of the slave device remains unchanged with respect to the nominal target pose speed module;
a non-linear function for speed values above said threshold speed value, in which the modified target pose speed module of the slave device is reduced with respect to the nominal target pose speed module.

8. A method according to claim 7, wherein said threshold speed value is between 0.015 m/s and 0.025 m/s.

9. A method according to claim 7, wherein said non-linear section of the speed transfer function has a trend tending to a horizontal asymptote defining a maximum speed of the slave device target.

10. A method according to claim 8, wherein said maximum speed value of the slave device target corresponds to a maximum speed module being reachable by the slave device.

11. A method according to claim 8, wherein a maximum speed of the slave device target is tunable.

12. A method according to claim 1, wherein the transfer function that modifies the translational speed of the modified target pose is dependent on the nominal target pose speed and on a virtual distance between the position of the nominal target pose and the current position of the slave device.

13. A method according to claim 12, wherein said transfer function is a virtual distance transfer function, and is a continuous and monotonous non-decreasing function, defined as:
a linear function, for virtual distance values below a predetermined threshold distance value, in which the modified target pose speed module of the slave device remains unchanged with respect to the nominal target pose speed module;
a non-linear function for virtual distance values above said threshold distance value, in which the modified target pose speed module of the slave device is reduced, with respect to the nominal target pose speed module, by an amount given by a transfer function of said virtual distance.

14. A method according to claim 13, wherein said threshold distance value is between 0.5 mm and 5 mm.

15. A method according to claim 13, wherein the virtual distance transfer function is a continuous, monotonous non-decreasing function, having the value “virtual distance+maximum distance” as an asymptote, where the parameter “maximum distance” is a tunable parameter defining a maximum allowed virtual distance between the modified target pose and the slave device position.

16. A method according to claim 15, wherein said maximum virtual distance value is between 0.5 mm and 5 mm.

17. A method according to claim 1, wherein said master device is a groundless-type master device;
and/or wherein said master device is a master device mechanically unconstrained to an operating console.

18. A robotic system for medical or surgical teleoperation, comprising:
at least one master device adapted to be moved by an operator;
at least one slave device comprising a surgical instrument adapted to be controlled by the master device;
a control unit configured to control the slave device, during a teleoperation, based on motions of the master device,
wherein the control unit is further configured to:
define a nominal target pose in a workspace of the slave device, corresponding to a respective pose of the master device in a workspace of the master device;
modify said nominal target pose to obtain a modified target pose of the slave device;
control motion of the slave device in the slave device workspace so that the slave device is configured to follow said modified target pose during a teleoperation;
wherein, in said step of modifying the nominal target pose to obtain the modified target pose, the control unit is configured to:
decrease a translational speed module of the modified target pose, with respect to a speed of the nominal target pose, according to a transfer function dependent on instantaneous speed of the master device and/or a distance between a current position of the slave device and the nominal target pose of the slave device,
and/or decrease instantaneous power or energy imparted by the master device to the slave device, according to a transfer function dependent on the instantaneous speed of the master device and/or the instantaneous power or energy of the master device and/or the distance between a current position of the slave device and the nominal target pose of the slave device.

19. (canceled)

20. A method according to claim 1, wherein said master device is a groundless-type master device without force feedback;
and/or wherein said master device is a master device of the type which is mechanically unconstrained to an operating console.
